(19) United States
(12) Patent Application Publication
(10) Pub. No.: US 2023/0221326 A1
(43) Pub. Date: Jul. 13, 2023

(54) Methods for Reducing Nonspecific Interactions on Biological Samples

(71) Applicant: Ultivue, Inc., Cambridge, MA (US)

(72) Inventors: Gourab Chatterjee, Cambridge, MA (US); Mael Manesse, Medford, MA (US)

(73) Assignee: Ultivue, Inc., Cambridge, MA (US)

(21) Appl. No.: 17/788,779

(22) PCT Filed: Dec. 29, 2020
(86) PCT No.: PCT/US2020/067295

§ 371 Date: Jun. 24, 2022

Related U.S. Application Data
(60) Provisional application No. 62/954,868, filed on Dec. 30, 2019.

(51) Int. Cl. G01N 33/58; G01N 33/53; C12Q 1/6804

(52) U.S. Cl. CPC G01N 33/58; C12Q 1/6804; G01N 33/5306

(57) ABSTRACT

This application describes various improved embodiments for detecting at least one target in tissue samples using an antibody-barcode conjugate capable of binding to the target and blocker strands to prevent nonspecific interaction of the antibody-barcode conjugate with non-target nucleic acid material. Some embodiments employ a blocker strand partially or fully complementary to a barcode portion of the antibody-barcode conjugate. Some embodiments employ a double-stranded barcode portion of an antibody-barcode conjugate, including a blocker strand.

### CROSS-REFERENCE TO RELATED APPLICATIONS

This application claims the benefit of priority of US Provisional Application No. 62/954,868, filed Dec. 30, 2019, which is incorporated by reference herein in its entirety for any purpose.

### SEQUENCE LISTING

This application is filed with a Sequence Listing in electronic format. The Sequence Listing is provided as a file entitled “01168-0021-00PCT_Seq_List_ST25.txt” created on Dec. 22, 2020, which is 8,192 bytes in size. The information in the electronic format of the sequence listing is incorporated herein by reference in its entirety.

### INTRODUCTION AND SUMMARY

This application relates to methods of detecting one or more targets in biological samples. In particular, this application relates to methods of detecting one or more targets in tissue samples using antibody-barcode conjugates.

Antibody-barcode conjugates are employed in a variety of tests for detecting molecular targets in biological materials. The antibody portion of a conjugate recognizes and binds selectively to its molecular target, while the barcode portion is used for generating a detectable signal. However, use of nucleic acid strands for signal detection or generation is often susceptible to the problem of nonspecific interaction with nucleic acids in the biological material. The extent of these nonspecific interactions can depend upon the assay setup; length and concentration of nucleic acid sequences used; the specific sequence composition, and other factors. These problematic nonspecific interactions can be reduced by using blocking reagents (e.g., BSA), or relatively inert nucleic acid sequences (e.g., salmon sperm DNA), which mask nucleic acid binding sites on the biological material and impede weaker interactions. These strategies may, however, fail to tackle more specific nonspecific interactions such as binding between partially complementary nucleic acid sequences and endogenous nuclear material in biological material. Therefore, strategies to prevent sequence-specific nonspecific interactions would be helpful for improving quality of these tests.

In accordance with the present disclosure, the following numbered embodiments are provided.

Embodiment 1. A method for detecting a target in a biological sample, comprising:
a) contacting a sample with an antibody-barcode conjugate comprising an antibody capable of binding to a target, and a barcode comprising single-stranded nucleic acid; and a blocker strand hybridized to the barcode, the blocker strand being selected from:
   a blocker strand that hybridizes to the barcode over the entire length of both the barcode and the blocker strand; or
   a blocker strand that hybridizes to the barcode over its entire length and further comprises a 5’ or 3’ toehold overhang;
(b) removing the blocker strand from the barcode; and
(c) detecting the antibody-barcode conjugate bound to the target using the barcode.

Embodiment 2. A method for detecting a target in a biological sample, comprising:
a) contacting a sample with an antibody-barcode conjugate comprising an antibody capable of binding to a target and a barcode comprising single-stranded nucleic acid; and a blocker strand hybridized to the barcode, wherein the blocker strand is a partially complementary blocker strand that hybridizes to the barcode over a first portion, but not over its entire length;
(b) removing the blocker strand from the barcode; and
(c) detecting the antibody-barcode conjugate bound to the target using the barcode.

Embodiment 3. The method of embodiment 2, wherein the partially complementary blocker strand hybridizes at the 5’ end of the barcode.

Embodiment 4. The method of embodiment 2, wherein the partially complementary blocker strand is hybridizes at the 3’ end of the barcode

Embodiment 5. A method for detecting two or more targets in a biological sample, comprising:
a) contacting a sample with two or more antibody-barcode conjugates, each comprising an antibody capable of binding to a different target and a distinct barcode comprising single-stranded nucleic acid; and a blocker strand hybridized to each distinct barcode, the blocker strand being selected from:
   a blocker strand that hybridizes to the barcode over the entire length of both the barcode and the blocker strand; or
   a blocker strand that hybridizes to the barcode over its entire length and further comprises a 5’ or 3’ toehold overhang;
(b) removing the blocker strands from the barcodes; and
(c) detecting the antibody-barcode conjugates bound to the targets using the barcodes.

Embodiment 6. A method for detecting a target in a biological sample using an antibody-barcode conjugate, comprising:
(a) contacting a sample with an antibody-barcode conjugate comprising an antibody capable of binding to a target, and a barcode precursor, wherein the barcode precursor comprises a double-stranded nucleic acid region comprising the barcode hybridized to a segment of a blocker strand;
(b) removing the blocker strand from the barcode precursor to form a single-stranded barcode linked to the antibody; and
(c) detect the antibody-barcode conjugate bound to the target using the barcode.

Embodiment 7. A method for detecting two or more targets in a biological sample, comprising:
a) contacting a sample with two or more antibody-barcode conjugates, each comprising an antibody capable of binding to a different target and a barcode precursor, wherein the barcode precursor comprises a double-stranded nucleic acid region comprising a distinct barcode hybridized to a segment of a blocker strand;
(b) removing the blocker strands from the barcode precursors to form single-stranded barcodes; and
(c) detecting the antibody-barcode conjugates bound to the targets using the barcodes.

Embodiment 8. The method of embodiment 6 or 7, wherein a portion of the blocker strand is single stranded.

Embodiment 9. The method of any one of embodiments 6-8, wherein the barcode precursor comprises a hairpin structure.

Embodiment 10. The method of embodiment 9, wherein the barcode precursor comprises one or more single-stranded loop domains.

Embodiment 11. The method of embodiment 9 or 10, wherein the barcode precursor comprises one or more double-stranded stem domains.

Embodiment 12. The method of any one of embodiments 1-11, wherein (c) detecting comprises binding an imager strand to the barcode.

Embodiment 13. The method of any one of embodiments 1-11, wherein (c) detecting comprises amplifying the barcode to produced amplified barcodes, and binding imager strands to the amplified barcodes.

Embodiment 14. The method of any one of embodiments 1-13, wherein (b) removing the blocker strand from the barcode is accomplished by strand dissociation.

Embodiment 15. The method of embodiment 14, wherein the strand disassociation is accomplished by increased temperature, low ionic strength solution, denaturant, dissociating agent, chemical cleavage, photochemical cleavage, and enzymatic cleavage, or a combination of one or more of these conditions.

Embodiment 16. The method of embodiment 14 or 15, wherein the strand disassociation is accomplished by increasing temperature prior to the removing step.

Embodiment 17. The method of any one of embodiments 14-16, wherein the strand disassociation is accomplished by contacting the sample with a dissociation solution.

Embodiment 18. The method of any one of embodiments 14-17, wherein the strand displacement is accomplished by contacting the sample with a dissociation solution and increasing temperature prior to the removing step.

Embodiment 19. The method of embodiment 18, wherein the dissociation solution comprises deionized water and/or low ionic strength solution.

Embodiment 20. The method of any one of embodiments 14-19, wherein the strand disassociation is accomplished by contacting the sample with one or more hydrophobic agent.

Embodiment 21. The method of embodiment 20, wherein the one or more hydrophobic agent comprises polyethylene glycol (PEG) or diglyme.

Embodiment 22. The method of any one of embodiments 14-21, wherein the strand disassociation is accomplished by contacting the sample with one or more dissociating agent.

Embodiment 23. The method of embodiment 22, wherein the one or more dissociating agent comprises an ethylene glycol ether.

Embodiment 24. The method of any one of embodiments 1-5 and 12-23, wherein (b) removing the blocker strand from the barcode is cleavage of the blocker strand chemically, photochemically, or enzymatically.

Embodiment 25. The method of any one of embodiments 6-23, wherein (b) removing the blocker strand from the barcode precursor is cleavage of the blocker strand chemically, photochemically, or enzymatically.

Embodiment 26. The method of embodiment 24 or 25, wherein the blocker strand comprises one or more cleavable site, and wherein (b) removing the blocker strand comprises cleaving the cleavable site.

Embodiment 27. The method of embodiment 26, wherein the one or more cleavable site is selected from: a chemical cleavage site; a mechanical cleavage site; a radiation cleavage site; and an enzymatic cleavage site.

Embodiment 28. The method of embodiment 26 or 27, wherein the one or more cleavable site comprises one or more of: disulfide bond (cleaved by reducing agents such as dithiothreitol or tris(2-carboxyethyl)phosphine)), ester (cleaved by hydroxylamine), vicinal diol (cleaved by sodium meta-periodate), sulfone (cleaved under basic conditions), photocleavable bond (cleaved by light), and a bond that can be cleaved enzymatically.

Embodiment 29. The method of any one of embodiments 26-28, wherein the enzymatic cleavage is accomplished by contacting the sample with an enzyme selected from glycosylase, endonuclease, exonuclease, DNAzyme, and deoxyribozyme.

Embodiment 30. The method of any one of embodiments 26-29, wherein (b) removing the blocker strand from the barcode is cleavage of a photocleavable site.

Embodiment 31. The method of embodiment 30, wherein the photocleavable site comprises o-nitrobenzyl (ONB) ester, α-thioacetophenone moiety, and 7-amino coumarin moiety.

Embodiment 32. The method of any one of embodiments 26-30, wherein the one or more cleavable site comprises disulfide bond and (b) removing the blocker strand comprises contacting the sample by a reducing agent to cleave the disulfide bond.

Embodiment 33. The method of embodiment 32, wherein the reducing agent comprises tris-carboxyethyl phosphine (TCEP).

Embodiment 34. The method of any one of embodiments 26-33, wherein the one or more cleavable site comprises a deoxyuridine.

Embodiment 35. The method of any one of embodiments 26-34, wherein the one or more cleavable site comprises a uracilated nucleotide and (b) removing the blocker strand comprises contacting the sample with Uracil-DNA glycosylase (UDG).

Embodiment 36. The method of any one of embodiments 26-34, wherein the one or more cleavable site comprises a uracilated nucleotide and (b) removing the blocker strand comprises contacting the sample with Uracil-DNA glycosylase (UDG) and Endonuclease VIII.

Embodiment 37. The method of any one of embodiments 26-34, wherein the one or more cleavable site comprises an abasic site with an intact phosphodiester backbone and (b) removing the blocker strand comprises contacting the sample with Endonuclease VIII.

Embodiment 38. The method of any one of embodiments 26-37, wherein the one or more cleavable site comprises a photocleavable linker and (b) removing the blocker strand comprises cleaving the cleavable linker by UV exposure.

Embodiment 39. The method of any one of embodiments 1-5 and 12-38, wherein (b) removing the blocker strand from the barcode is accomplished by strand displacement.

Embodiment 40. The method of embodiment 39, wherein the strand displacement is accomplished by contacting the sample with a complementary nucleic acid strand (or a displacement strand).

Embodiment 41. The method of any one of embodiments 1-5 and 12-40, wherein the blocker strand includes a 5’ or 3’ toehold overhang, and wherein (b) removing the blocker strand from the barcode comprises contacting the sample with a displacement strand that binds to the 5’ or 3’ toehold overhang of the blocker strand and denatures the hybridization of the blocker strand and the barcode to release the blocker strand.

Embodiment 42. The method of any one of embodiments 1-41, wherein the blocker strand is single stranded.

Embodiment 43. The method of any one of embodiments 1-41, wherein the blocker strand is partially double stranded.

Embodiment 44. The method of any one of embodiments 1-43, wherein the barcode has a length of from 5 to 50 nucleotides, or from 8 to 15 nucleotides, or from 10 to 13 nucleotides.

Embodiment 45. The method of any one of embodiments 1-44, wherein the barcode is linked at a 5’ end to the antibody portion.

Embodiment 46. The method of any one of embodiments 1-45, wherein (c) detecting further comprises contacting the sample with an imager strand capable of specifically binding the barcode, wherein the imager strand linked to a detectable label; and detecting signal from the detectable label linked to the imager strand.

Embodiment 47. The method of embodiment 46, wherein the method further comprises removing unbound imager strand.

Embodiment 48. The method of embodiment 46 or 47, wherein the imager strand is a single-stranded nucleic acid.

Embodiment 49. The method of any one of embodiments 46-48, wherein the label is a fluorophore.

Embodiment 50. The method of any one of embodiments 46-48, wherein the label is a metal particle, plasmonic enhancer, enzyme (e.g., HRP), primer, capture oligonucleotide or splint of rolling circle amplification, or initiator of finite or infinite hybridization chain reaction.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1B show schematic representation of certain aspects of the present disclosure. FIG. 1A shows a schematic representation of nonspecific interactions between antibody-barcode conjugates and endogenous nucleic acids in a tissue sample. FIG. 1B shows a schematic representation of an exemplary embodiment for reducing nonspecific interactions of antibody-barcode conjugates with endogenous nucleic acids in a tissue sample.

FIG. 2 shows an exemplary embodiment for reducing nonspecific interactions in tissue staining involving partially blocking the barcode in antibody-barcode conjugates and subsequently unblocking the barcode using strand dissociation or displacement.

FIG. 3 shows an exemplary embodiment for reducing nonspecific interactions in tissue staining involving completely blocking the barcode in antibody-barcode conjugates and subsequently unblocking the barcode using cleavage of the blocker strand.

FIG. 4 shows an exemplary embodiment for reducing nonspecific interactions in tissue staining involving blocking the barcode in antibody-barcode conjugates and subsequently unblocking the barcode through toehold-mediated strand displacement.

FIG. 5 shows an exemplary embodiment for reducing nonspecific interactions in tissue staining involving using an antibody-barcode conjugate containing a hairpin structure, and cleaving away a segment to generate a single-stranded portion.

FIGS. 6A-6C show images of tissue staining in accordance with an embodiment described herein in the absence of blocking (FIG. 6A) and in the presence of partial blocking (FIG. 6B) and complete blocking (FIG. 6C).

FIGS. 7A-7C show images of tissue staining in accordance with an embodiment described herein without using blocker strands (FIG. 7A) and with using blocker strands followed by removal of the blocker strands (FIG. 7B), with quantitative comparison on the background signal levels (FIG. 7C).

FIGS. 8A-8C show images of tissue staining in accordance with an embodiment described herein without using blocker strands (FIG. 8A) and with using blocker strands followed by removal of the blocker strands (FIG. 8B), with quantitative comparison on the background signal levels (FIG. 8C).

### DESCRIPTION OF THE EMBODIMENTS

The methods and compositions described herein involve using antibody-barcode conjugates for detecting targets. The antibody portion of the conjugate binds selectively to the target, and the barcode conjugate portion is typically an element of a detection system. However, significant barcode nucleotide sequence complementarity-mediated nonspecific interactions can take place due to partial complementarity of the single-stranded barcode in the conjugate with nucleic acid molecules in the sample to be tested, such as endogenous nuclear material (DNA/RNA) in biological samples. Such interactions can lead to nonspecific signal (background) in these assays. The methods and compositions described herein are useful for reducing nonspecific signal when using antibody-barcode conjugates for detecting the presence or amount of a target in a biological sample. The methods and compositions described herein can be used for detecting one or more targets sequentially or simultaneously.

### I. Definitions

As used herein, the term “nucleic acid” refers to a polymeric form of nucleotides of any length, such as deoxyribonucleotides or ribonucleotides, or analogs thereof. A “nucleic acid” includes, without limitation, DNA, RNA, cDNA, aptamers, peptide nucleic acids (“PNA”) and locked nucleic acids (“LNA”). A nucleic acid may comprise modified nucleotides, such as methylated nucleotides and nucleotide analogs (“analogous” forms of purines and pyrimidines are well known in the art). A nucleic acid also may comprise also include an altered phosphate backbone, an altered pentose sugar, and/or altered nucleobases. A nucleic acid also may include, but are not limited to, 2’-O-Methyl ribonucleic acid, 2’-fluoro ribonucleic acid, peptide nucleic acid, morpholino and locked nucleic acid, glycol nucleic acid, and threose nucleic acid. If present, modifications to the nucleotide structure may be imparted before or after assembly of the polymer. In various embodiments, a nucleic acid may be a single-stranded, double-stranded, partially single-stranded, or partially double-stranded nucleic acid.

As used herein, the term “barcode” means a nucleic acid strand distinct for a target of interest and being conjugated to an antibody capable of binding to a target of interest.

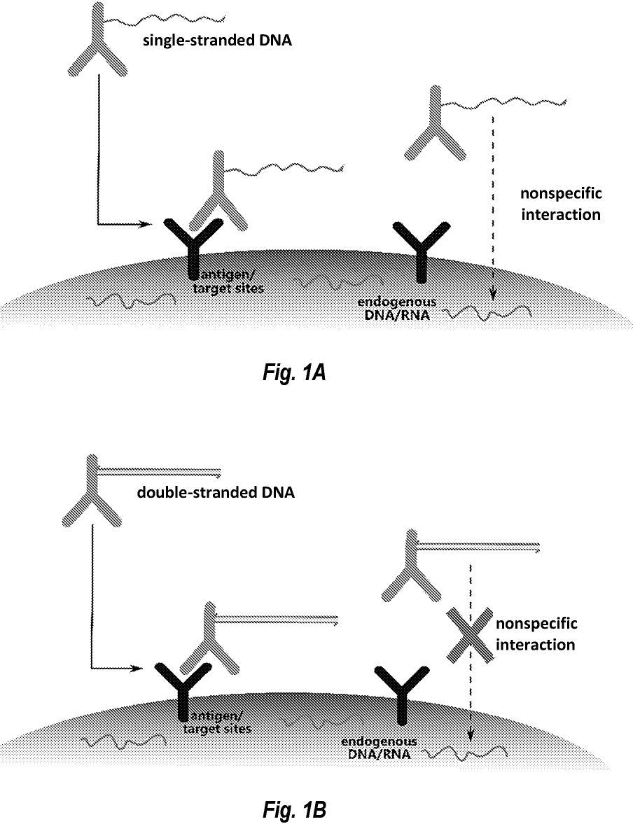

As used herein, the term “blocker strand” means a nucleic acid strand that binds specifically to the barcode portion of an antibody-barcode conjugate. When bound to a barcode, it blocks association with any off-target nucleic acids (see FIG. 1B). A blocker strand can be partially complementary towards the 5’-end or the 3’-end of the barcode portion, leaving a shorter single-stranded domain; or can be fully complementary. The blocker strand can be removed from the barcode portion, for example, by displacement or dissociation.

As used herein, the term “cleavage site” means a structure within a nucleic acid portion of a barcode or antibody-barcode conjugate that is susceptible to the action of its corresponding cleavage agent. Thus, the molecular characteristics of a cleavage site depend on its corresponding cleavage agent (described further below).

As used herein, the term “imager strand” means a nucleic acid strand capable of specifically binding either directly or through an intermediate strand, to a barcode, and linked to a detectable label (e.g., fluorophore) that emits a signal and can be used to detect the presence of a target of interest.

As used herein, the term “antibody” encompasses both full length antibodies and any antibody-like molecules that include any engineered variants or fragments of an antibody such as Fab, Fab′, F(ab′)2, single heavy chain, diabody, and the like that can specifically recognize a target (e.g., antigen binding fragments of antibodies). Antibody variants may contain one or more modifications (e.g., an amino acid insertion, deletion, substitution, a post-translational modification or lack thereof, etc.).

As used herein, the term “target” means the antigen to which an antibody used in a method described herein selectively binds. A target can be, for example, one or more biomolecules, including but not limited to large macromolecules and polymers such as proteins, carbohydrates, lipids, nucleic acids, as well as small molecules such as primary metabolites, secondary metabolites, and natural products.

As used herein, the term “nonspecific” when used in reference to a barcode means unwanted binding of a substance present in a sample to the barcode. A barcode as described herein is used for generating a detectable signal corresponding to the presence or amount of a target. Therefore, when a barcode binds nonspecifically to one or more components of a sample, such as a natural biological material of the sample (e.g., endogenous nucleic acid having sufficient complementarity to bind the barcode); an additive material (e.g., fixative, assay component, etc.), including components of the sample environment (e.g., solid support such as a slide, other assay component), this unwanted binding can interfere with the intended barcode function and cause undesirable results..

### II. Methods for Detection of Targets

In the methods described herein, single-stranded barcode of an antibody-barcode conjugate is blocked via complementary “blocker strands” such that the resultant double-stranded barcode is not available for nonspecific interaction with endogenous substances of a biological sample (e.g., nucleic acid). In the methods, the barcode portion of the conjugate is in a blocked configuration when the antibody portion of the conjugate binds to its target. After target binding, the single-stranded barcode portion is restored by unblocking. This results in reduced nucleic acid mediated nonspecific interactions of the conjugates, without detrimental effect on subsequent assay steps.

FIG. 1A shows a schematic representation of an antibody-barcode conjugate binding to a tissue sample. In this representation, the barcode portion of the conjugate is single stranded. The antibody portion binds to its target and the barcode portion is available for binding to a labeled probe (e.g., an imager strand) or other detection scheme. Binding of the barcode portion to endogenous nucleic acid in the tissue is depicted as a nonspecific interaction. FIG. 1B shows a schematic representation according to certain embodiments of the present disclosure of blocking this nonspecific interaction by binding a nucleic acid strand to the barcode portion, rendering it unavailable for binding to endogenous nucleic acid.

In some embodiments, a method for detecting a target in a biological sample is provided, the method comprising contacting a sample with an antibody-barcode conjugate comprising an antibody capable of binding to a target, and a barcode comprising single-stranded nucleic acid; and a blocker strand hybridized to the barcode, and detecting the antibody-barcode conjugate bound to the target. In some embodiments, the blocker strand hybridizes to the barcode over the entire length of both the barcode and the blocker strand. In some embodiments, the blocker strand is a particially complementary blocker strand that hybridizes to the barcode over a first portion, but not over its entire length. In some embodiments, the antibody-barcode conjugate bound to the target is detected by removing the blocker strand from the barcode and using binding of the unblocked barcode to a labeled imager strand. In some embodiments, the antibody-barcode conjugate bound to the target is detected by binding of the antibody-barcode conjugate to a secondary antibody linked to a detectable label (such as enzyme-(e.g., HRP)-mediated labeling). In this embodiment, removal of the blocker strand from the barcode is optional.

In some embodiments, a method for detecting a target in a biological sample is provided, the method comprising: (a) contacting a sample with an antibody-barcode conjugate comprising an antibody capable of binding to a target, and a barcode comprising single-stranded nucleic acid; and a blocker strand hybridized to the barcode, the blocker strand being selected from: a blocker strand that hybridizes to the barcode over the entire length of both the barcode and the blocker strand; or a blocker strand that hybridizes to the barcode over its entire length and further comprises a 5’ or 3’ toehold overhang; (b) removing the blocker strand from the barcode; and (c) detecting the antibody-barcode conjugate bound to the target using the barcode.

In some embodiments, a method for detecting a target in a biological sample using an antibody-barcode conjugate is provided, the method comprising: (a) contacting a sample with an antibody-barcode conjugate comprising an antibody capable of binding to a target and a barcode comprising single-stranded nucleic acid; and a blocker strand hybridized to the barcode, wherein the blocker strand is a partially complementary blocker strand that hybridizes to the barcode over a first portion, but not over its entire length; (b) removing the blocker strand from the barcode; and (c) detecting the antibody-barcode conjugate bound to the target using the barcode. In some embodiments, the partially complementary blocker strand hybridizes at the 5’ end of the barcode. In some embodiments, the partially complementary blocker strand hybridizes at the 3’ end of the barcode.

In some embodiments, a method for detecting a target in a biological sample using an antibody-barcode conjugate is provided, the method comprising: (a) contacting a sample with an antibody-barcode conjugate comprising an antibody capable of binding to a target, and a barcode precursor, wherein the barcode precursor comprises a double-stranded nucleic acid region comprising the barcode hybridized to a segment of a blocker strand; (b) removing the blocker strand from the barcode precursor to form a single-stranded barcode linked to the antibody; and (c) detect the antibody-barcode conjugate bound to the target using the barcode. In some embodiments, a portion of the blocker strand is single stranded. In some embodiments, the barcode precursor comprises a hairpin structure. In some embodiments, the barcode precursor comprises one or more single-stranded loop domains. In some embodiments, the barcode precursor comprises one or more double-stranded stem domains.

In some embodiments, removing the blocker strand from the barcode or the barcode precursor is accomplished by strand dissociation. In some embodiments, strand disassociation is accomplished by increased temperature, low ionic strength solution, denaturant, dissociating agent, chemical cleavage, photochemical cleavage, and enzymatic cleavage, or a combination of one or more of these conditions. In some embodiments, removing the blocker strand from the barcode or the barcode precursor is accomplished by strand displacement.

In some embodiments, wherein detecting comprises binding an imager strand to the barcode. In some embodiments, wherein detecting comprises amplifying the barcode to produced amplified barcodes, and binding imager strands to the amplified barcodes. In some embodiments, detecting comprises contacting the sample with an imager strand capable of specifically binding to the barcode, wherein the imager strand linked to a detectable label; and detecting signal from the detectable label linked to the imager strand. In some embodiments, the method further comprises removing unbound imager strand.

In some embodiments, a method for detecting a target in a biological sample using an antibody-barcode conjugate is provided, the method comprising: (a) contacting a sample with two or more antibody-barcode conjugates, each comprising an antibody capable of binding to a different target and a distinct barcode comprising single-stranded nucleic acid; and a blocker strand hybridized to each distinct barcode, the blocker strand being selected from a blocker strand that hybridizes to the barcode over the entire length of both the barcode and the blocker strand; or a blocker strand that hybridizes to the barcode over its entire length and further comprises a 5’ or 3’ toehold overhang; (b) removing the blocker strands from the barcodes; and (c) detecting the antibody-barcode conjugates bound to the targets using the barcodes.

In some embodiments, a method for detecting a target in a biological sample using an antibody-barcode conjugate is provided, the method comprising: (a) contacting a sample with two or more antibody-barcode conjugates, each comprising an antibody capable of binding to a different target and a barcode precursor, wherein the barcode precursor comprises a double-stranded nucleic acid region comprising a distinct barcode hybridized to a segment of a blocker strand; (b) removing the blocker strands from the barcode precursors to form single-stranded barcodes; and (c) detecting the antibody-barcode conjugates bound to the targets using the barcodes.

In some embodiments, the barcode comprises DNA. In some embodiments, a method for detecting a target in a biological sample using an antibody-DNA conjugate is provided, the method comprising: (a) contacting a sample with an antibody-DNA conjugate under conditions wherein the antibody portion of the antibody-DNA conjugate can bind to its target, wherein the antibody-DNA conjugate comprises a single-stranded DNA portion, and a feature selected from: a partially overlapping blocker strand, bound to the single-stranded DNA portion; a completely overlapping blocker strand, bound to the single-stranded DNA portion; or a toehold blocker strand, bound to the single-stranded DNA portion; (b) removing the blocker strand; and (c) using the single-stranded DNA portion to detect the antibody-DNA conjugate.

In some embodiments, a method for detecting a target in a biological sample using an antibody-DNA conjugate is provided, the method comprising: (a) contacting a sample with an antibody-DNA conjugate under conditions wherein the antibody portion of the antibody-DNA conjugate can bind to its target, wherein the antibody-DNA conjugate comprises a double-stranded DNA portion; (b) cleaving the blocker strand to generate a single-stranded DNA portion; and (c) using the single-stranded DNA portion to detect the antibody-DNA conjugate.

### A. Blocker Strand

In some embodiments, the methods described herein comprise contacting a sample with an antibody-barcode conjugate comprising an antibody capable of binding to a target and a barcode comprising single-stranded nucleic acid; and a blocker strand hybridized to the barcode. In some embodiments, the blocker strand hybridizes to the barcode over the entire length of both the barcode and the blocker strand. In some embodiments, the blocker strand hybridizes to the barcode over its entire length and further comprises a 5’ or 3’ toehold overhang. In some embodiments, the blocker strand is a partially complementary blocker strand that hybridizes to the barcode over a first portion, but not over its entire length.

In some embodiments, a blocker strand can be partially complementary towards the 5’-end or the 3’-end of the barcode portion, leaving a shorter single-stranded domain; or can be fully complementary. The blocker strand can be removed from the barcode portion, for example, by displacement or dissociation.

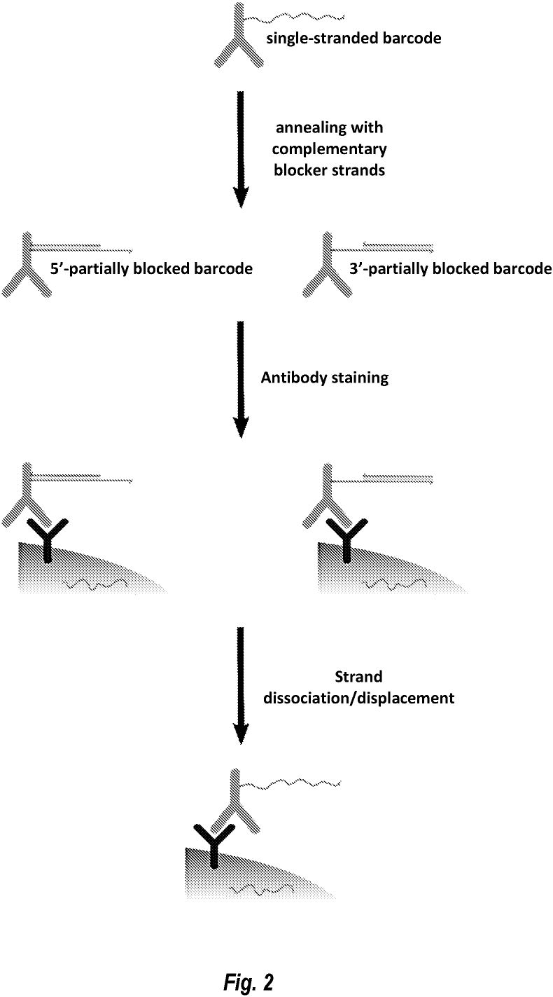

FIG. 2 shows a schematic representation according to certain embodiments of the present disclosure of staining a tissue sample using an antibody-barcode conjugate annealed to a complementary blocker strand that is partially complementary towards the 5’-end or the 3’-end of the barcode portion. Once the antibody-barcode conjugate is bound to the target, the blocker strand is removed by dissociation or displacement. Accordingly, in some embodiments, the blocker strand hybridizes at the 5’ end of the barcode. In some embodiments, the blocker strand hybridizes at the 3’ end of the barcode.

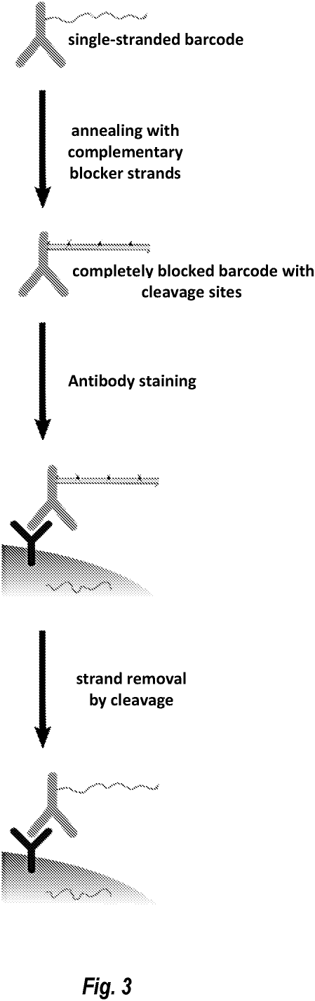

FIG. 3 shows a schematic representation according to certain embodiments of the present disclosure of staining a tissue sample using an antibody-barcode conjugate annealed to a blocker strand that is fully complementary to the barcode over its entire length. Once the antibody-barcode conjugate is bound to the target, the blocker strand is removed by dissociation (cleavage).

In some embodiments, the method further comprises removing blocker strands not bound to the antibody-barcode conjugate. Unbound blocker strands may be removed in a washing step (e.g., PBS with 0.1% Tween-20).

### B. Strand Displacement

In some embodiments, the methods described herein comprise removing the blocker strand from the barcode by strand displacement.

Strand displacement can be accomplished by contacting the conjugate with a complementary nucleic acid strand. For example, contact with a fully complementary strand will typically result in dissociation of the blocker strand.

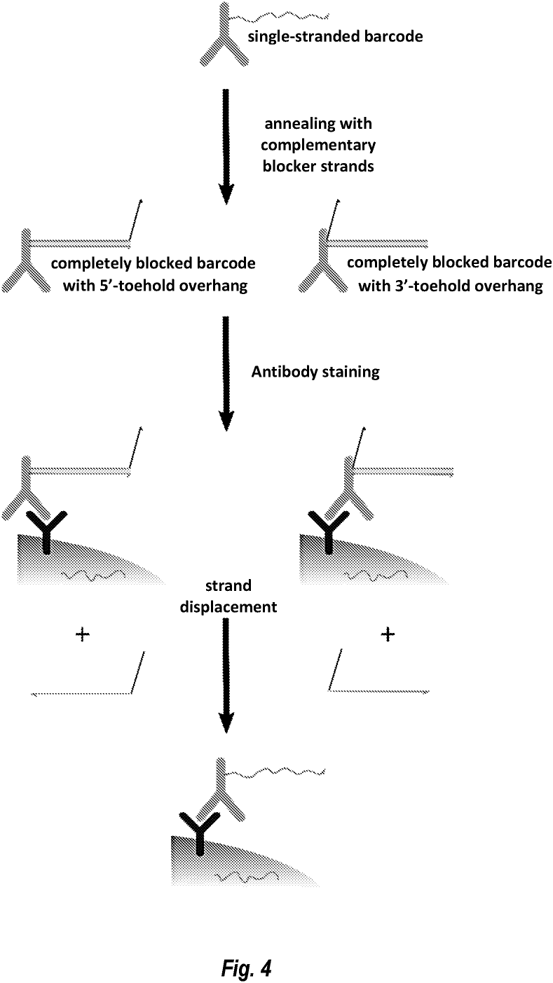

FIG. 4 shows a schematic representation of staining a tissue sample using an antibody-barcode conjugate annealed to a complementary blocker strand containing a 5’ or 3’ toehold overhang. The single-stranded toehold domains are useful for subsequent removal of the blocker strands via toehold-mediated strand displacement.

Toehold-mediated strand displacement is a method for the isothermal and dynamic exchange of DNA complexes. Strand displacement can be designed and controlled based on an understanding of DNA hybridization interactions and thermodynamics and can be facilitated by introducing engineered handles which are known as “toehold domains.”. Accordingly, in some embodiments, the strand displacement is accomplished by contacting the sample with a complementary nucleic acid strand (or a displacement strand). In some embodiments, the blocker strand includes a 5’ or 3’ toehold domain. In some embodiments, the removing the blocker strand from the barcode comprises: contacting the sample with a displacement strand that binds to the 5’ or 3’ toehold overhang of the blocker strand and denatures the hybridization of the blocker strand and the barcode to release the blocker strand.

### C. Strand Disassociation

In some embodiments, the methods disclosed herein involve removing the blocker strand from the barcode by strand dissociation.

Strand dissociation can be accomplished by physical conditions such as temperature and wash solution composition. Strand dissociation methods include, but are not limited to, wash steps with a dissociation solution such as deionized water and/or low-salt solution at elevated temperatures; deionized water with optimized concentrations of hydrophobic agents like polyethylene glycol (PEG), diglyme, etc. (destabilizes double helix conformation); and other well-known conditions for dissociating nucleic acid strands. The blocker strand can also be removed by cleavage with a cleavage agent. Such a blocker strand will contain one or more cleavage sites for the selected cleavage agent.

In some embodiments, the strand disassociation is accomplished by increased temperature, low ionic strength solution, denaturant, dissociating agent, chemical cleavage, photochemical cleavage, and enzymatic cleavage, or a combination of one or more of these conditions. Increasing the temperature or lowering the ionic strength of the solution containing the nucleic acid strands disrupts the binding affinities between the nucleic acid strands. As used herein, “increased temperature” or “increasing temperature” means incubating the sample at and/or subjecting the sample with washes of temperatures higher than ambient temperature and up to about 95° C. for specific periods of time. Strand dissociation can be facilitated by incubating the sample in a solution at high temperatures for up to 30 minutes, followed by replenishment with fresh solution and repeating the incubation step if required. As used herein, “low ionic strength” means the ionic strength of the medium measured by the amount of salt concentration in the medium being less than 300 mM. In an embodiment, the salt concentration is below the concentration required for the Tm to be lower than the ambient temperature, e.g., lower than 70 mM. The hybridization rate decreases with lower salt concentration. Dehybridization at low ionic strength may be achieved by conventional methods, for example, reducing the salt (e.g., Na+) concentration by adding water.

In some embodiments, the strand disassociation is accomplished by increasing temperature prior to the removing step. In some embodiments, the strand disassociation is accomplished by contacting the sample with a dissociation solution. In some embodiments, the dissociation solution comprising deionized water and/or low ionic strength solution.

In some embodiments, the strand disassociation is accomplished by contacting the sample with one or more hydrophobic agent. In some embodiments, the one or more hydrophobic agent is selected from polyethylene glycol (PEG) and diglyme.

In some embodiments, the strand disassociation is accomplished by contacting the sample with one or more dissociating agent. In some embodiments, the one or more dissociating agent comprises an ethylene glycol ether.

In some embodiments, an antibody-barcode conjugate comprises a barcode precursor, wherein the barcode precursor comprises a double-stranded nucleic acid region comprising the barcode hybridized to a segment of a blocker strand. The barcode precursor may have a hairpin structure. The barcode precursor may include one or more single-stranded loop domains. The barcode precursor also may include one or more stem domains.

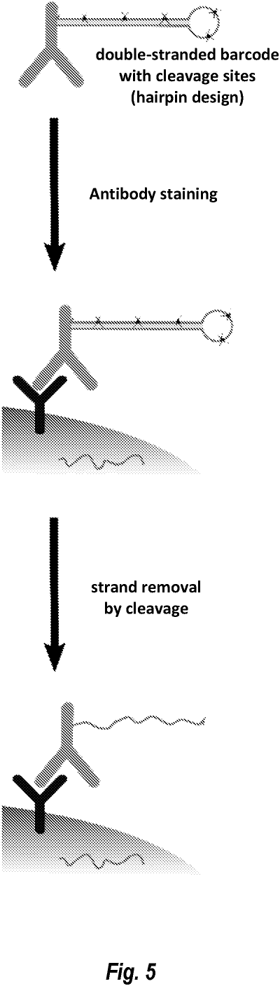

In some embodiments, the barcode precursor of an antibody-barcode conjugate contains a double-stranded region. For example, FIG. 5 shows a schematic representation of an antibody-barcode conjugate containing a hairpin-shaped barcode precursor modified with cleavage sites that allow nucleic acid strand cleavage (represented as black star-like shapes in FIG. 5). The cleavage sites are present at the hairpin loop and the trailing strand (3′-end) of the hairpin stem region.

### D. Cleavage Site

In some embodiments, the methods described herein comprise removing the blocker strand from the barcode or the barcode precursor is cleavage of the blocker strand chemically, photochemically, or enzymatically, or a combination of one or more thereof.

Cleavage sites can include (but are not limited to) uracilated nucleotides (for USER/UDG-mediated exchange), di-sulfide linkers (for chemical reduction using DTT/TCEP), photo-labile linkers (for photo-cleavage), specific recognition sites for site-specific endonuclease digestion and other non-enzymatic cleavage mechanisms. Depending upon the position of the cleavage sites, the resultant cleaved strands can spontaneously dissociate from the conjugate and are optionally washed off during wash steps post-cleavage. The resultant single-stranded barcode portion can be used for signal generation and/or detection. For example, FIG. 3 shows blocker strand removal by cleavage of cleavage sites contained within the blocker strand or the blocker strand duplex.

As used herein, the term “cleavage site” means a structure within a blocker strand that is susceptible to the action of its corresponding cleavage agent. Thus, the molecular characteristics of a cleavage site depend on its corresponding cleavage agent. Accordingly, a cleavage site can be selected from a chemical cleavage site; a mechanical cleavage site; a radiation cleavage site; an enzymatic cleavage site. Exemplary chemical bonds that can be cleaved are disulfide bonds (cleaved by reducing agents such as dithiothreitol or tris (2-carboxyethyl)phosphine)), esters (cleaved by hydroxylamine), vicinal diols (cleaved by sodium meta-periodate), sulfones (cleaved under basic conditions), photocleavable bonds (cleaved by light), and bonds that can be cleaved using enzymes such as hydrolases, nucleases, uracil DNA glycosylase, and DNA glycosylase-lyase Endonuclease VIII, (e.g., USER (Uracil-Specific Excision Reagent) (New England Biolabs)). Non-natural nucleotides that serve as substrates for particular enzymes can be used in a cleavage site. For example, 8-oxoguanine may be cleaved by DNA glycosylase OGG1. For example, a 1′,2′-Dideoxyribose, dSpacer, apurinic/apyrimidinic, tetrahydrofuran, or abasic furan may be cleaved by Endonuclease VIII cleavage sites.

In some embodiments, the blocker strand comprises one or more disulfide bond. In some embodiments, the methods disclosed herein comprise contacting the sample with a reducing agent to cleave the disulfide bonds. In an embodiment, tris-carboxyethyl phosphine (TCEP) may be used as a reducing agent.

In some embodiments, a radiation cleavage site is a photocleavage site, which is cleaved by the presence of light of a particular spectral range. A variety of chemical bonds are susceptible to photocleavage (see, for example, Olejnik et al., Nucleic Acids Res. 1999 Dec 1;27(23):4626-31; and Leriche et al. Bioorganic & Medicinal Chemistry, volume 20(2), 571-582 (2012).) Among well-known photocleavable moieties include o-nitrobenzyl (ONB) esters, α-thioacetophenone moieties, and 7-amino coumarin moieties. See, for example, CRC Handbook of Organic Photochemistry and Photobiology, 2nd Edition, chapter 69. Exemplary photocleavable moieties that can be incorporated into oligonucleotides are commercially available through Biosynthesis, Inc., Lewisville, TX; Integrated DNA Technologies, Skokie, IL and other companies. In some embodiments, the blocker strand comprises one or more photocleavable site. In some embodiments, the methods described herein comprise contacting the sample with UV exposure to remove the blocker strand. The photocleavable site may comprise o-nitrobenzyl (ONB) ester, α-thioacetophenone moiety, and 7-amino coumarin moiety.

A variety of enzymatically cleavable moieties can be used in a method or composition described herein. A number of enzymes can break the covalent bonds within a nucleic acid molecule. For example, glycosylases can remove a base from the sugar moiety of a nucleotide; endonucleases, exonuclease, DNAzymes, and deoxyribozymes can cleave phosphodiester bonds of nucleic acid molecules, and enzymes can be engineered for cleaving at a cleavage site.

A glycosylase capable of specifically removing a base that participates in nucleotide base-pairing can reduce the strength of interaction between the two strands. For example, deoxyuridine (dU) can be substituted for deoxythymidine (dT) at a cleavage site; dU would pair with dA, and this pair would be cleaved by Uracil-DNA Glycosylase (UDG, commercially available from New England Biolabs, Cat #M0280S). This reaction will result in abasic site(s) at the cleavage site. Such abasic sites can be further cleaved by Endonuclease VIII, or another method. This promotes dissociation of remnant binding pairs. UDG or a combination of UDG and Endonuclease VIII can therefore be useful in performing the methods described herein. A mixture of these enzymes is commercially available (e.g., from New England Biolabs, under the tradename USER, Cat# M5505S).

When designing a UDG cleavage site, a number of dU nucleotides will be present, ranging from 1 to 5 dUs; 1 to 10 dUs; 1 to 15 dUs; and 1 to 20 dUs. When using UDG and Endonuclease VIII, the dUs can be placed in a way that, after removal of dU, the remnants are short (e.g., less than or equal to about 9, 8, 7, 6, 5, 4, 3, 2, or 1 nucleotides) such that they dissociate spontaneously and relatively quickly.

Accordingly, in some embodiments, the blocker strand comprises one or more uracilated nucleotide as a cleavage site. In some embodiments, the methods described herein comprise contacting the sample with Uracil-DNA glycosylase (UDG) to remove the blocker strand. In some embodiments, the methods described herein comprise contacting the sample with Uracil-DNA glycosylase (UDG) and Endonuclease VIII to remove the blocker strand. In some embodiments, the blocker strand comprises one or more abasic site with an intact phosphodiester backbone as a cleavage site. In some embodiments, the methods described herein comprise contacting the sample with Endonuclease VIII to remove the blocker strand.

Endonucleases having site specific activity useful in the methods described herein include restriction endonucleases, zinc finger nucleases, transcription activator-like effector nucleases (TALENs), and deoxyribozymes.

An RNA guided endonuclease can be used in methods described herein. For example, Cas9 (CRISPR associated protein 9) is an RNA-guided endonuclease can specifically cleave an engineered cleavage site. One strand may be cleaved, for example using a nicking endonuclease. As an example, Cas9 nickases are Cas9 enzymes that have been engineered to only include one active cleaving site, leading to single strand cuts, while conserving the high specificity of Cas9.

Accordingly, a variety of cleavage agents are useful in the methods described herein. A cleavage agent can therefore be a chemical agent, enzymatic agent, radiation (e.g., UV light, visible light, infrared, near infrared, x-ray, microwave, radio waves, gamma rays), mechanical force, or any other agent that renders at least a segment of the barcode portion of the conjugate single stranded.

### E. Exemplary Antibody-Barcode Conjugates

Antibody-barcode conjugates have been described, for example, in US 2018/0164308A1, which is incorporated herein by reference.

Exemplary DNA barcode sequences and corresponding complementary sequences are shown in Table 1.

TABLE 1

| DNA portion sequence | SEQ ID NO | Complementary sequence | SEQ ID NO |
|---|---|---|---|
| 5′-ACGGAACCAACA–3′ | 1 | 5′-TGTTGGTTCCGT–3′ | 13 |
| 5′-ACGGAATGAGGC–3′ | 2 | 5′-GCCTCATTCCGT–3′ | 14 |
| 5′-ACTTGCTGACGA–3′ | 3 | 5′-TCGTCAGCAAGT–3′ | 15 |
| 5′-TCACGTCAGCAT–3′ | 4 | 5′-ATGCTGACGTGA–3′ | 16 |
| 5′-TTGACGATGGCA–3′ | 5 | 5′-TGCCATCGTCAA–3′ | 17 |
| 5′-GGGAAGTAGGGC–3′ | 6 | 5′-GCCCTACTTCCC–3′ | 18 |
| 5′-CCCAAAACGTCG–3′ | 7 | 5′-CGACGTTTTGGG–3′ | 19 |
| 5′-TCGCTGTCATGA–3′ | 8 | 5′-TCATGACAGCGA–3′ | 20 |
| 5′-AGCAATTCGGGT–3′ | 9 | 5′-ACCCGAATTGCT–3′ | 21 |
| 5′-CGGGTTAAGGGT–3′ | 10 | 5′-ACCCTTAACCCG–3′ | 22 |
| 5′-GCGTTGGGATGA–3′ | 11 | 5′-TCATCCCAACGC–3′ | 23 |
| 5′-AGCGAGGAAAGT–3′ | 12 | 5′-ACTTTCCTCGCT–3′ | 24 |

Tissue staining using antibody-barcode conjugates is well known to those skilled in the art. Antibody-barcode conjugates can be prepared as described, for example, in https://www.nature.com/articles/srep22675; https://journals.plos.org/plosone/article?id= 10.1371 /journal.pone.0209860; and elsewhere.

In some embodiments, the barcode comprises single-stranded nucleic acids and may be from about 5 to 50 nucleic acids long, from about 8 to 15, or from about 10 to 13 nucleic acids long. In some embodiments, the barcode is about 30 to 50 nucleic acids long. In some embodiments, the barcode is about 5, 8, 9, 10, 11, 12, 13, 14, 15, 18, 20, 25, 30, 34, 38, 42, 46 or 50 nucleic acids long.

In some embodiments, an antibody-barcode conjugate contains a hairpin-shaped structure including a barcode hybridized to a segment of a blocker strand (FIG. 5). The cleavage sites are present at the hairpin loop and the trailing strand (3′-end) of the hairpin stem region. After cleavage, the 5′ end of the hairpin stem region conjugated to the antibody serves as a barcode.

### F. Exemplary Imager Strand

A variety of imager strands may be used. In some embodiments, the imager strand may be a nucleic acid strand that is complementary to the barcode and is linked to a detectable label. In other words, the imager strand specifically binds the barcode. In some embodiments, the imager strand may be from about 5 to 50 nucleic acids long, from about 8 to 15, or from about 10 to 12 nucleic acids long.

An imager strand may have one or more portions complementary to a barcode. In some embodiments, a complementary portion between the imager strand and the barcode may be from about 5 to 50 nucleic acids long, from about 8 to 15, or from about 10 to 12 nucleic acids long nucleic acids long. In some embodiments, a complementary portion between the imager strand and the barcode may be about 5, 8, 9, 10, 11, 12, 13, 14, 15, 18, or 20 nucleic acids long.

In some embodiments, the barcode may bind to the imager strand indirectly, such as through an intermediate moiety. For instance, an intermediate moiety may comprise a first region complementary to the barcode and a second region complementary to the imager strand. In this embodiment, it is not necessary for the barcode to be complementary to the imager strand. The intermediate moiety may serve only a bridging function or it may also serve an amplification function.

Detectable labels, such as optical labels, may comprise fluorophores. Any selection of fluorophores can be used so long as the signals from each sample can be distinguished. The measurement can be, for example, prompt fluorescence, fluorescence polarization, fluorescence resonance energy transfer (FRET), or time-resolved fluorescence. Fluorophores that can be used in combination with each other, may include (one set per dye channel), for example: AL405 channel (Alexa405, ATTO390, Alexa350); AT488 channel (Alexa488, ATTO488, FITC); AT565 channel (ATTO565, Alexa565, TAMRA, TRITC), lanthanides and quantum dots. Signals from the labels may be measured using a fluorescence reader, such as a multi-mode plate reader, a flow cytometer, capillary reader, and a fluorescent microscope or scanner.

### G. Amplification

Various nucleic acid amplification methods may be employed to amplify the barcode portion of the antibody-barcode conjugate to produce amplified barcodes. Depending upon the nucleic acid amplification method employed, the amplified barcodes may have the same sequence as the barcode sequence or its complementary sequence. Thus, the amplified barcodes may have a different sequence from the barcode portion of the antibody-barcode conjugate.

For example, in rolling circle amplification (RCA), a barcode portion of the antibody-barcode conjugate is amplified using an amplifier strand (in some instances a circular nucleic acid template), followed by extension of the barcode by a DNA polymerase to create a concatemeric repeat of the reverse complement of the amplifier strand (i.e., an amplified barcodes). Accordingly, in some embodiments, detecting comprises amplifying the barcode to produced amplified barcodes, and binding labelled imager strands to the amplified barcodes, wherein the amplified barcodes comprise a concatemer of the sequence complementary to the barcode.

Primer Exchange Reaction (PER) may also be used as an amplification strategy. PER can be used to prepare a nucleic acid product containing multiple sites for binding imager strands (i.e., intermediate moiety described herein); this nucleic acid product can then be bound to the barcode of the antibody-barcode conjugate Various PER and PER-based signal amplification methods have been described in Saka et al., “Highly multiplexed in situ protein imaging with signal amplification by Immuno-SABER” (2018; available as a preprint at www.biorxiv.org/content/10.1101/507566v1 as of Jun. 6, 2019); WO 2017/143006; and WO 2018/132392A2, the contents of each of which are herein incorporated by reference for their teaching of PER signal amplification methods. In some embodiments, the PER reaction results in the formation of concatemer (repeat) sequences. The PER concatemer has a first domain that is complimentary to the barcode of the antibody-barcode conjugate and a second domain comprising repeat sequences. The repeated sequence may be the same as, or different from, the barcode of the antibody-barcode conjugate (or a portion of the barcode) and can be used in a detection scheme, for example, for binding imager strands

Other examples of nucleic acid amplification methods include, but are not limited to, hybridization chain reaction (HCR) (Dirks et al., 2014, PMID: 15492210, 24712299), a similar hairpin-based dendrimerization reaction (HDR) (Yin et al., 2008, PMID 18202654), branched toehold-based strand displacement (Schweller et al. PMCID: PMC3517005), the contents of each of which are herein incorporated by reference in their entirety for the teachings of each nucleic acid amplification method.

### H. Samples

As used herein, the term “sample” means any natural or man-made biological fluid, cell, tissue, or fraction thereof, or other material, that includes or is suspected to include a target. A sample can be derived from a prokaryote or eukaryote and therefore can include cells from, for example, animals, plants, or fungi. Accordingly, a sample includes a specimen obtained from one or more individuals or can be derived therefrom. For example, a sample can be a tissue section (e.g., obtained by biopsy), or cells that are placed in or adapted to tissue culture. Exemplary samples include biological specimens such a cheek swab, amniotic fluid, skin biopsy, organ biopsy, tumor biopsy, blood, urine, saliva, semen, sputum, cerebral spinal fluid, tears, mucus, and the like. A sample can be further fractionated, if desired, to a fraction containing particular cell types. For example, a blood sample can be fractionated into serum or into fractions containing particular types of blood cells. If desired, a sample can be a combination of samples from an individual such as a combination of a tissue and fluid.

When used in a method described herein, a sample can be fixed to a surface. Exemplary surfaces include a slide, a plate, a bead, a tube, and a capillary. Prior to analysis, a sample can be processed to preserve the integrity of targets. Such methods include the use of appropriate buffers and/or inhibitors, including nuclease, protease and phosphatase inhibitors, that preserve or minimize changes in the molecules in the sample. Methods for preserving tissue samples are well known and include fixatives. The particular preservation method selected will depend sample type.

In some embodiments, the sample is a cell, cell lysate, tissue, tissue lysate, a bodily fluid and/or a whole organism. In some embodiments, the sample is a tissue sample.

### EXAMPLES

### Example 1

The effect of partial and complete blocking of DNA in antibody-DNA “barcode” conjugates on the background signal generated due to nonspecific interactions of those conjugates with tissue sections were examined using diaminobenzidine (DAB) staining.

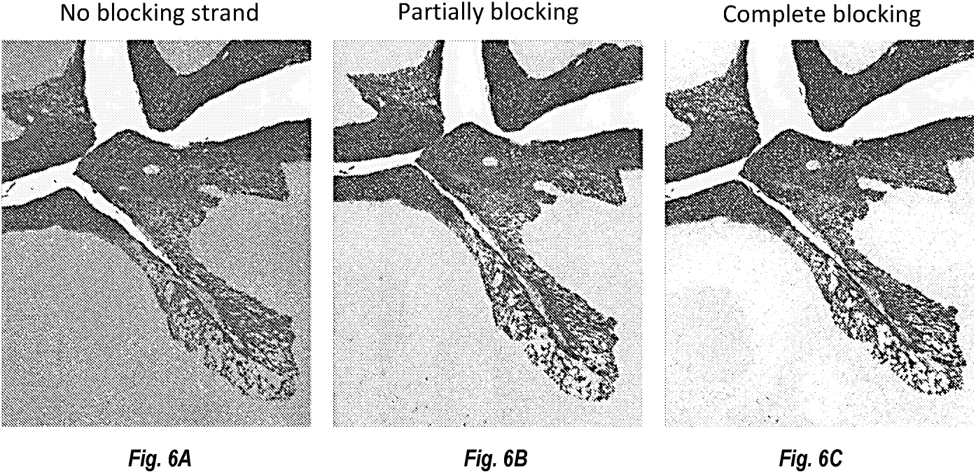

Briefly, serial sections of FFPE human tonsil tissue were stained with pan-cytokeratin antibody-barcode conjugates under different conditions. The conditions include antibody-barcode conjugates with: unblocked “barcode” DNA strands (FIG. 6A), barcodes partially blocked with partially complementary blocker strands (FIG. 6B), and barcodes completely blocked with fully complementary blocker strands (FIG. 6C). Post staining, the interaction of these antibody-barcode conjugates to cytokeratin in the tonsil tissue sections were detected using the commercially available diaminobenzidine-based (DAB) biomarker detection assay with hematoxylin nuclear counter-stain (BOND Polymer Refine Detection Kit, Leica Biosystems). Using this method, a polymeric horseradish peroxidase (HRP)-linker antibody was bound to the anti-cytokeratin antibody of the conjugate. The HRP enzyme catalyzes oxidation of DAB by hydrogen peroxide. The oxidized DAB forms a brown precipitate, at the location of the HRP, which was visualized using light microscopy.

FIGS. 6A-6C show images of DAB tissue staining of cytokeratin using pan-cytokeratin antibody-barcode conjugates. A specific cytokeratin staining pattern is observed around the epithelial regions including cytokeratin. Regions apart from the epithelial regions, including in and around the germinal centers are expected to not have any specific signal due to absence of cytokeratin. Those regions should exhibit minimal signal (white background) in the absence of any nonspecific interaction of the conjugates with the tissue sections. Any faint gray signal in these regions signify nonspecific interaction of the antibody-barcode conjugates with the tissue sections.

It was observed that use of a blocker strand that partially blocked the barcode reduced background signal, while use of a blocker strand that fully blocked the barcode further reduced background signal.

### Example 2

This example shows use of a blocker strand containing uracil groups to reduce nonspecific background signal.

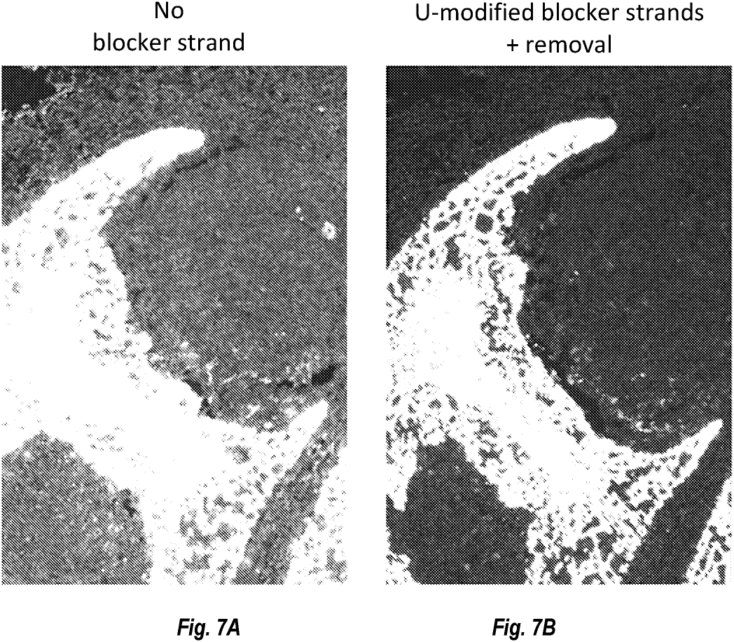

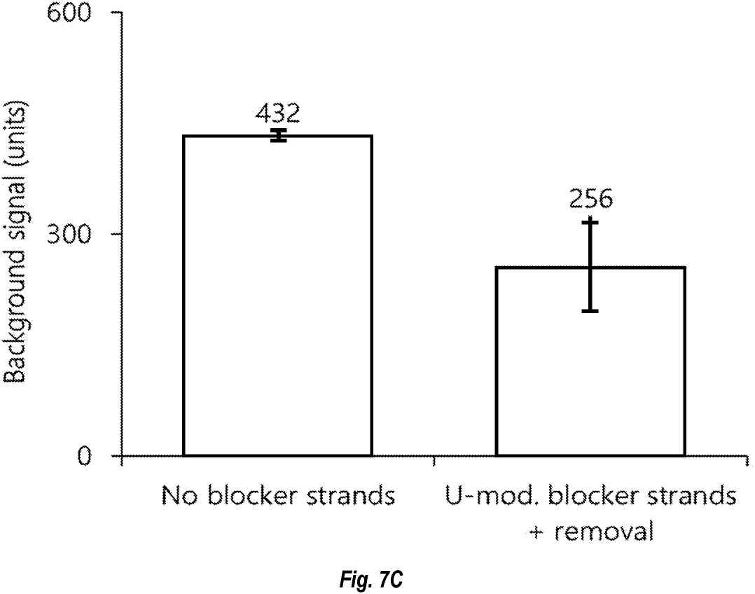

The effect of blocker strands with uracilated nucleotides as cleavage sites (U-modified blocker strands), towards reducing the nonspecific interaction of conjugates on tonsil tissue sections were evaluated. Briefly, serial sections of FFPE human tonsil tissue were stained with pan-cytokeratin antibody-barcode conjugates under different conditions, and the signal was detected using Ultivue ULTIMAPPER immunofluorescence assay. For ULTIMAPPER staining, following incubation of a FFPE tissue sample with antibody-barcode conjugates (“staining”), the sample is washed and incubated with detection reagents whereby the barcode of the antibody-barcode conjugate is amplified. Fluorescently labeled imager strands are then bound to the amplified bar codes; the sample is washes, and the fluorescent signal is detected using a fluorescence microscope. FIGS. 7A-7B show representative areas of tonsil tissue sections with cytokeratin positive cells as well as nonspecific background signal from cytokeratin negative cells. The serial sections were stained with pan-cytokeratin antibody-barcode conjugates either with no blocker strands (FIG. 7A), or after a brief pre-hybridization step with U-modified blocker strands (FIG. 7B). After conjugate staining, the blocker strands were removed via a brief strand removal step (as depicted in FIG. 3). Briefly, the blocker strands were subjected to an USER-mediated cleavage step targeting the uracilated bases in the blocker strands. This was followed by a gentle wash step to facilitate the dissociation of cleaved strands, thereby exposing the single-stranded barcode. Fluorescence signal corresponds to the white portion of the photograph, such that the darker background shown in FIG. 7B corresponds to less nonspecific fluorescent staining. FIG. 7C shows a quantitative comparison on the background signal levels due to nonspecific interaction of conjugates in the absence or presence of the U-modified blocker strands.

It was observed that use of a blocker strand reduced nonspecific background signal.

### Example 3

This example shows use of a blocker strand containing disulfide groups to reduce nonspecific background signal.

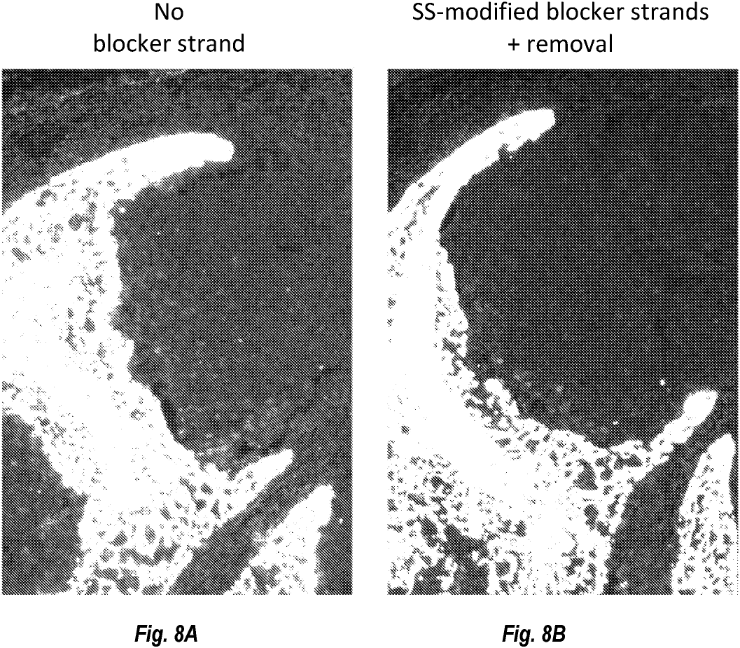

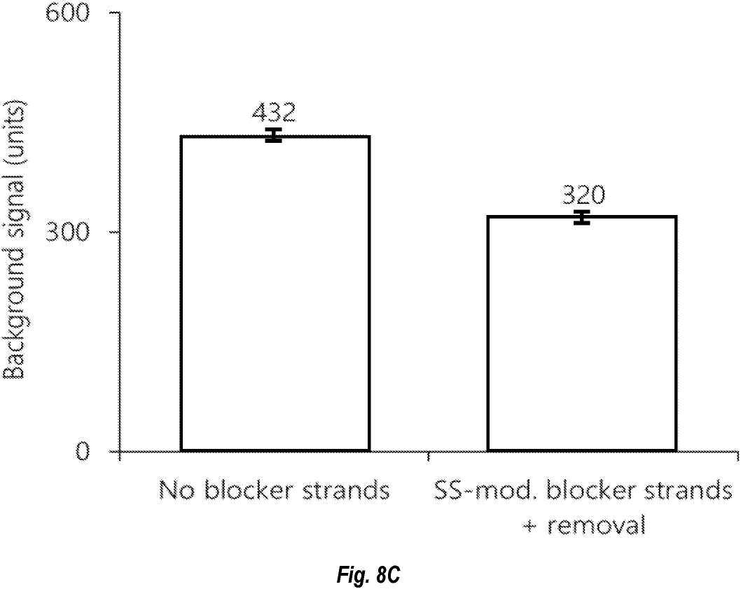

The effect of blocker strands with disulfide spacers as cleavage sites (SS-modified blocker strands), towards reducing the nonspecific interaction of conjugates on tonsil tissue sections were evaluated. Briefly, serial sections of FFPE human tonsil tissue were stained with pan-cytokeratin antibody-barcode conjugates under different conditions, and the signal was detected using Ultivue ULTIMAPPER immunofluorescence assay (described in Example 2). FIGS. 8A-8B show representative areas of tonsil tissue sections with cytokeratin positive cells as well as nonspecific background signal from cytokeratin negative cells. The serial sections were stained with pan-cytokeratin antibody-barcode conjugates either with no blocker strands (FIG. 8A), or after a brief pre-hybridization step with SS-modified blocker strands (FIG. 8B). After conjugate staining, the blocker strands were removed via a brief strand removal step (as depicted in FIG. 3). Briefly, the blocker strands were subjected to a TCEP-mediated cleavage step targeting the disulfide-modified bases in the blocker strands. This was followed by a gentle wash step to facilitate the dissociation of cleaved strands, thereby exposing the single-stranded barcode. Fluorescence signal corresponds to the white portion of the photograph, such that the darker background shown in FIG. 8B corresponds to less nonspecific fluorescent staining. FIG. 8C shows a quantitative comparison on the background signal levels due to nonspecific interaction of conjugates in the absence or presence of the SS-modified blocker strands.

It was observed that use of a blocker strand reduced nonspecific background signal.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term “about” refers to a numeric value, including, for example, whole numbers, fractions, and percentages, whether or not explicitly indicated. The term about generally refers to a range of numerical values (e.g., +/-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method for detecting a target in a biological sample, comprising:
a) contacting a sample with an antibody-barcode conjugate comprising an antibody capable of binding to a target, and a barcode comprising single-stranded nucleic acid; and a blocker strand hybridized to the barcode, the blocker strand being selected from:
a blocker strand that hybridizes to the barcode over the entire length of both the barcode and the blocker strand; or
a blocker strand that hybridizes to the barcode over its entire length and further comprises a 5’ or 3’ toehold overhang;
(b) removing the blocker strand from the barcode; and
(c) detecting the antibody-barcode conjugate bound to the target using the barcode.

2. A method for detecting a target in a biological sample using an antibody-barcode conjugate, comprising:
(a) contacting a sample with an antibody-barcode conjugate comprising an antibody capable of binding to a target, and a barcode precursor, wherein the barcode precursor comprises a double-stranded nucleic acid region comprising the barcode hybridized to a segment of a blocker strand;
(b) removing the blocker strand from the barcode precursor to form a single-stranded barcode linked to the antibody; and
(c) detect the antibody-barcode conjugate bound to the target using the barcode.

3. The method of claim 2, wherein a portion of the blocker strand is single stranded.

4. The method of claim 2 or 3, wherein the barcode precursor comprises a hairpin structure.

5. The method of claim 4, wherein the barcode precursor comprises one or more single-stranded loop domains.

6. The method of claim 4 or 5, wherein the barcode precursor comprises one or more double-stranded stem domains.

7. The method of any one of claims 1-6, wherein (c) detecting comprises binding an imager strand to the barcode.

8. The method of any one of claims 1-6, wherein (c) detecting comprises amplifying the barcode to produced amplified barcodes, and binding imager strands to the amplified barcodes.

9. The method of any one of claims 1-8, wherein (b) removing the blocker strand from the barcode is accomplished by strand dissociation.

10. The method of claim 9, wherein the strand disassociation is accomplished by increased temperature, low ionic strength solution, denaturant, dissociating agent, chemical cleavage, photochemical cleavage, and enzymatic cleavage, or a combination of one or more of these conditions.

11. The method of claim 9 or 10, wherein the strand disassociation is accomplished by contacting the sample with one or more dissociating agent.

12. The method of claim 11, wherein the one or more dissociating agent comprises an ethylene glycol ether.

13. The method of any one of claims 1 and 7-12, wherein (b) removing the blocker strand from the barcode is cleavage of the blocker strand chemically, photochemically, or enzymatically.

14. The method of any one of claims 2–12, wherein (b) removing the blocker strand from the barcode precursor is cleavage of the blocker strand chemically, photochemically, or enzymatically.

15. The method of claim 13 or 14, wherein the blocker strand comprises one or more cleavable site, and wherein (b) removing the blocker strand comprises cleaving the cleavable site.

16. The method of claim 15, wherein the one or more cleavable site comprises disulfide bond and (b) removing the blocker strand comprises contacting the sample by a reducing agent to cleave the disulfide bond.

17. The method of claim 15, wherein the one or more cleavable site comprises a uracilated nucleotide and (b) removing the blocker strand comprises contacting the sample with Uracil-DNA glycosylase (UDG).

18. The method of claim 15, wherein the one or more cleavable site comprises a photocleavable linker and (b) removing the blocker strand comprises cleaving the cleavable linker by UV exposure.

19. The method of any one of claims 1 and 7-18, wherein (b) removing the blocker strand from the barcode is accomplished by strand displacement.

20. The method of any one of claims 1-19, wherein the barcode has a length of from 5 to 50 nucleotides, or from 8 to 15 nucleotides, or from 10 to 13 nucleotides.

21. A method for detecting two or more targets in a biological sample, comprising:
a) contacting a sample with two or more antibody-barcode conjugates, each comprising an antibody capable of binding to a different target and a distinct barcode comprising single-stranded nucleic acid; and a blocker strand hybridized to each distinct barcode, the blocker strand being selected from:
a blocker strand that hybridizes to the barcode over the entire length of both the barcode and the blocker strand; or
a blocker strand that hybridizes to the barcode over its entire length and further comprises a 5’ or 3’ toehold overhang;
(b) removing the blocker strands from the barcodes; and
(c) detecting the antibody-barcode conjugates bound to the targets using the barcodes.

22. A method for detecting two or more targets in a biological sample, comprising:
a) contacting a sample with two or more antibody-barcode conjugates, each comprising an antibody capable of binding to a different target and a barcode precursor, wherein the barcode precursor comprises a double-stranded nucleic acid region comprising a distinct barcode hybridized to a segment of a blocker strand;
(b) removing the blocker strands from the barcode precursors to form single-stranded barcodes; and
(c) detecting the antibody-barcode conjugates bound to the targets using the barcodes.


SEQUENCE LISTING (24 sequences)

SEQ ID NO: 1 - DNA, 12 bp, Artificial sequence
  Exemplary DNA portion
acggaaccaaca

SEQ ID NO: 2 - DNA, 12 bp, Artificial sequence
  Exemplary DNA portion
acggaatgaggc

SEQ ID NO: 3 - DNA, 12 bp, Artificial sequence
  Exemplary DNA portion
acttgctgacga

SEQ ID NO: 4 - DNA, 12 bp, Artificial sequence
  Exemplary DNA portion
tcacgtcagcat

SEQ ID NO: 5 - DNA, 12 bp, Artificial sequence
  Exemplary DNA portion
ttgacgatggca

SEQ ID NO: 6 - DNA, 12 bp, Artificial sequence
  Exemplary DNA portion
gggaagtagggc

SEQ ID NO: 7 - DNA, 12 bp, Artificial sequence
  Exemplary DNA portion
cccaaaacgtcg

SEQ ID NO: 8 - DNA, 12 bp, Artificial sequence
  Exemplary DNA portion
tcgctgtcatga

SEQ ID NO: 9 - DNA, 12 bp, Artificial sequence
  Exemplary DNA portion
agcaattcgggt

SEQ ID NO: 10 - DNA, 12 bp, Artificial sequence
  Exemplary DNA portion
cgggttaagggt

SEQ ID NO: 11 - DNA, 12 bp, Artificial sequence
  Exemplary DNA portion
gcgttgggatga

SEQ ID NO: 12 - DNA, 12 bp, Artificial sequence
  Exemplary DNA portion
agcgaggaaagt

SEQ ID NO: 13 - DNA, 12 bp, Artificial sequence
  Exemplary DNA portion, complementary sequence to SEQ ID NO: 1
tgttggttccgt

SEQ ID NO: 14 - DNA, 12 bp, Artificial sequence
  Exemplary DNA portion, complementary sequence to SEQ ID NO: 2
gcctcattccgt

SEQ ID NO: 15 - DNA, 12 bp, Artificial sequence
  Exemplary DNA portion, complementary sequence to SEQ ID NO: 3
tcgtcagcaagt

SEQ ID NO: 16 - DNA, 12 bp, Artificial sequence
  Exemplary DNA portion, complementary sequence to SEQ ID NO: 4
atgctgacgtga

SEQ ID NO: 17 - DNA, 12 bp, Artificial sequence
  Exemplary DNA portion, complementary sequence to SEQ ID NO: 5
tgccatcgtcaa

SEQ ID NO: 18 - DNA, 12 bp, Artificial sequence
  Exemplary DNA portion, complementary sequence to SEQ ID NO: 6
gccctacttccc

SEQ ID NO: 19 - DNA, 12 bp, Artificial sequence
  Exemplary DNA portion, complementary sequence to SEQ ID NO: 7
cgacgttttggg

SEQ ID NO: 20 - DNA, 12 bp, Artificial sequence
  Exemplary DNA portion, complementary sequence to SEQ ID NO: 8
tcatgacagcga

SEQ ID NO: 21 - DNA, 12 bp, Artificial sequence
  Exemplary DNA portion, complementary sequence to SEQ ID NO: 9
acccgaattgct

SEQ ID NO: 22 - DNA, 12 bp, Artificial sequence
  Exemplary DNA portion, complementary sequence to SEQ ID NO: 10
acccttaacccg

SEQ ID NO: 23 - DNA, 12 bp, Artificial sequence
  Exemplary DNA portion, complementary sequence to SEQ ID NO: 11
tcatcccaacgc

SEQ ID NO: 24 - DNA, 12 bp, Artificial sequence
  Exemplary DNA portion, complementary sequence to SEQ ID NO: 12
actttcctcgct